(19) United States
(12) Patent Application Publication
(10) Pub. No.: US 2008/0306386 A1
(43) Pub. Date: Dec. 11, 2008

(54) ULTRASONIC DIAGNOSTIC APPARATUS AND METHOD OF MEASURING VELOCITY WITH ULTRASONIC WAVES

(76) Inventors: Tatsuro BABA, Otawara-shi (JP); Yoichi Ogasawara, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/132,383

(22) Filed: Jun. 3, 2008

(30) Foreign Application Priority Data
Jun. 5, 2007 (JP) 2007-149466

(51) Int. Cl. A61B 8/06

(52) U.S. Cl. 600/455

(57) ABSTRACT

A multiple ultrasonic beam is transmitted from an ultrasonic probe, and a velocity of blood flow, azimuth and elevation angle of a sample such as a blood flow are acquired by a three-dimensional information acquisition section, as three-dimensional fluid information in a range gate, based on a Doppler signal output from the ultrasonic probe.

### CROSS-REFERENCE TO RELATED APPLICATIONS

This application is based upon and claims the benefit of priority from prior Japanese Patent Application No. 2007-149466, filed Jun. 5, 2007, the entire contents of which are incorporated herein by reference.

### BACKGROUND OF THE INVENTION

1. Field of the Invention

The present invention relates to Doppler angle correction for obtaining an absolute value of a velocity of blood flow by measuring a Doppler angle. In particular, the present invention relates to an ultrasonic diagnostic apparatus, which measures a velocity of a sample such as fluid, for example, blood flowing in a living body such as a human body, and a method of measuring a velocity with ultrasonic waves.

2. Description of the Related Art

An ultrasonic Doppler diagnostic apparatus emits an ultrasonic beam into a living body, and receives a wave reflected from blood flowing in a blood vessel in a living body, for example. An ultrasonic Doppler diagnostic apparatus measures a velocity of blood flow by using the Doppler effect caused by a reflected wave frequency shifted slightly from an incident frequency when an ultrasonic beam is reflected from a blood flow.

However, as an ultrasonic Doppler diagnostic apparatus is influenced by an angle formed by a blood flow direction and a direction of a wave reflected from a blood flow, it is difficult to directly measure the velocity in the direction of blood flow. Namely, an ultrasonic Doppler diagnostic apparatus creates two-dimensional ultrasonic sectional image data based on a Doppler signal output from an ultrasonic probe, and displays the data in a display section. The user or doctor tries to determine a true value of a velocity of blood flow by Doppler angle correction based on the image data in the direction of a blood vessel included in the two-dimensional ultrasonic sectional image data. However, the velocity of blood flow obtained by the Doppler angle correction in the two-dimensional ultrasonic sectional image data is insufficient to compensate for the influence in the depth direction that is a three-dimensional direction. Thus, the velocity of blood flow obtained by the Doppler angle correction lacks reliability.

Doppler angle correction is usually performed by measuring a velocity of blood flow in a range gate (RG), or a part where the flow of blood is measured by emitting an ultrasonic beam by using a pulse Doppler (PWD) method. Doppler angle correction is not performed in color Doppler tomography.

Doppler angle correction is available in the following technologies. A first technology is Jorgen Arendet Jensen, “Estimation of blood velocities using ultrasound: A signal processing approach”, Cambridge University Pres, New York, 1996. In the first technology, a velocity of blood flow is measured in two dimensions. In the first technology, an ultrasonic beam transmitter-receiver 1 and a receiver 2 are provided at an angle φ₁₀ as shown in FIG. 20. A velocity of blood flow is obtained in two dimensions based on the angle φ₁₀ and the angles θ₁₀ and θ₁₁ formed by the transmitter-receiver 1 and receiver 2 in the direction of blood flow vector B. FIG. 21 is a schematic diagram showing the transmitter-receiver 1 and receiver 2. The drawing is called a Jensen model. Tx indicates a transmitter. Rx 1 and Rx 2 indicate a receiver. The receiver (Rx 2) receives a wave reflected from a blood vessel 12.

A second technology is Robin Steel and Peten J. Fish, “Error Propagation Bounds in Dual and Triple Bean Vecter Doppler Ultrasound”, IEEE TRANSACTIONS ON ULTRASONICS, FERROELECTRICS, AND FREQUENCY CONTROL, VOL, 49, no. 9, September 2002. The second technology discloses that a velocity of blood flow is measured in three dimensions. The second technology has a transmitter (Tx) 4 and two receivers (Rx 1 and Rx 2) 5 and 6. The transmitter (Tx) 4 transmits an ultrasonic beam to an area including a blood vessel 12. The receivers (Rx 1 and Rx 2) 5 and 6 receive waves reflected from the blood vessel 12.

It is an object of the present invention to provide an ultrasonic diagnostic apparatus, which can exactly obtain a velocity and direction of an example such as a blood flow, and a method of measuring a velocity with ultrasonic waves.

### BRIEF SUMMARY OF THE INVENTION

According to a first aspect of the invention, there is provided an ultrasonic diagnostic apparatus comprising an ultrasonic probe which transmits a multiple ultrasonic beam to a sample flowing in a specific area, and receives a wave reflected from each part set at very small intervals in the sample; and a three-dimensional information acquisition section which acquires three dimensional fluid information including at least a three-dimensional flow direction of the sample in the specific area based on a Doppler signal output from an ultrasonic probe.

According to a second aspect of the invention, there is provided an ultrasonic diagnostic apparatus comprising an ultrasonic probe which transmits a multiple ultrasonic beam to a sample flowing in a specific area, and receives waves reflected from parts adjacent to each other in the sample in the specific area; and a three-dimensional information acquisition section which acquires three dimensional fluid information including at least a three-dimensional flow direction of the sample in the specific area based on a Doppler signal output from the ultrasonic probe.

### BRIEF DESCRIPTION OF THE SEVERAL VIEWS OF THE DRAWING

FIG. 1 is a block diagram of a first embodiment of an ultrasonic Doppler diagnostic apparatus according to the invention;

FIG. 2 is a schematic diagram for explaining estimation of a direction and magnitude of blood flow by the same apparatus;

FIG. 3 is a diagram showing a direction of a received beam and a direction of a blood vessel for explaining estimation of a direction and magnitude of blood flow by the same apparatus;

FIG. 4 is a block diagram showing a second embodiment of an ultrasonic Doppler diagnostic apparatus according to the invention;

FIG. 5 is a schematic diagram of a two-dimensional surface of an ultrasonic probe in the same apparatus;

FIG. 6 is a view for explaining a method of Doppler angle correction applied to the same apparatus;

FIG. 7 is a schematic diagram for explaining estimation of a direction and magnitude of blood flow by the same apparatus;

FIG. 8 is a schematic diagram for explaining Doppler angle correction on a two-dimensional cross section in the same apparatus;

FIG. 9 is a schematic diagram for explaining a method of Doppler angle correction applied to the same apparatus;

FIG. 10 is a schematic diagram for explaining a method of Doppler angle correction applied to the same apparatus;

FIG. 11 is a schematic diagram for explaining a method of Doppler angle correction applied to the same apparatus;

FIG. 12 is a block diagram of a reception delay adder circuit using a delay circuit in the same apparatus;

FIG. 13 is a is a block diagram of a reception delay adder circuit using a multiplier in the same apparatus;

FIG. 14 is a is a block diagram of a reception delay adder circuit combining a delay circuit and a multiplier in the same apparatus;

FIG. 15 is a diagram showing the result of error evaluation simulation for the estimation of the velocity of an example such as a blood flow for the same apparatus;

FIG. 16A is a diagram for explaining an example of using an ultrasonic oscillator in the same apparatus;

FIG. 16B is diagram for explaining an example of using an ultrasonic oscillator in the same apparatus;

FIG. 17A is diagram for explaining an example of using an ultrasonic oscillator in the same apparatus;

FIG. 17B is diagram for explaining an example of using an ultrasonic oscillator in the same apparatus;

FIG. 18A is diagram for explaining an example of using an ultrasonic oscillator in the same apparatus;

FIG. 18B is diagram for explaining an example of using an ultrasonic oscillator in the same apparatus;

FIG. 19A is diagram for explaining an example of using an ultrasonic oscillator in the same apparatus;

FIG. 19B is diagram for explaining an example of using an ultrasonic oscillator in the same apparatus;

FIG. 20 is a layout of a transmitter-receiver and a receiver for explaining conventional Doppler angle correction;

FIG. 21 is a schematic diagram showing the same transmitter-receiver and receiver; and

FIG. 22 is a schematic diagram showing a transmitter-receiver and a receiver for explaining another conventional Doppler angle correction.

### DETAILED DESCRIPTION OF THE INVENTION

Hereinafter, an explanation will be given on a first embodiment of the invention with reference to the accompanying drawings.

FIG. 1 is a block diagram of an ultrasonic Doppler diagnostic apparatus. An ultrasonic probe 10 emits a multiple ultrasonic beam pulse consisting of a plurality of beams to a specific area including a sample 13 (hereinafter called a range gate RG), and receives a wave reflected from the range gate RG. The sample 13 is fluid such as blood flowing in a blood vessel 12 in a living body 11 such as a human body. The ultrasonic probe 10 is composed of ultrasonic oscillators arranged on a two-dimensional plane. The ultrasonic probe 10 transmits a multiple ultrasonic beam, and receives waves reflected from the ultrasonic oscillators.

A scanning transmitter-receiver 14 electrically scans the ultrasonic oscillators of the ultrasonic probe 10, thereby sequentially driving the ultrasonic oscillators and outputting a multiple ultrasonic beam for scanning. The scanning transmitter-receiver 14 detects a Doppler signal from each output signal from each ultrasonic oscillator when receiving a wave reflected from the range gate RG.

A digital scan converter (hereinafter called a DSC) 15 converts the Doppler signal output from the scanning transmitter-receiver 14, and stores such signal in a storage 16 such as an image memory. The DSC 15 reads the digital Doppler signal stored in the storage 16 according to the scanning of a display 17, converts such signal to an analog signal, and displays an ultrasonic image of the range gate RG in the display 17. The ultrasonic image of the range gate RG includes the sample 13 such as blood flowing in the blood vessel 12 in the living body 11 such as a human body. The DSC 15 has a three-dimensional image data creation section 18, a three-dimensional information acquisition section 19, and a display section 20. The DSC 15 is connected to the display 17.

The three-dimensional image data creation section 18 converts the Doppler signal output from the scanning transmitter-receiver 14 to a digital signal, and stores a digital Doppler signal for a preset scanning period in the storage 16, thereby obtaining a plurality of sectional image data (stack data). By reconfiguring the obtained sectional image data, the three-dimensional data creation section 18 creates three-dimensional ultrasonic image data (volume data) of the range gate RG including the sample 13 such as blood flowing in the blood vessel 12 in the living body 11.

The three-dimensional information acquisition section 19 acquires three-dimensional fluid information including at least three-dimensional flow direction of the sample 13 included in the three-dimensional ultrasonic image data of a specific area, or the range gate RG, created by the three-dimensional data creation section 18. The three-dimensional information acquisition section 19 acquires three-dimensional fluid information based on the direction and magnitude of each reflected wave received by the ultrasonic probe 10 based on each Doppler signal output from the ultrasonic probe 10.

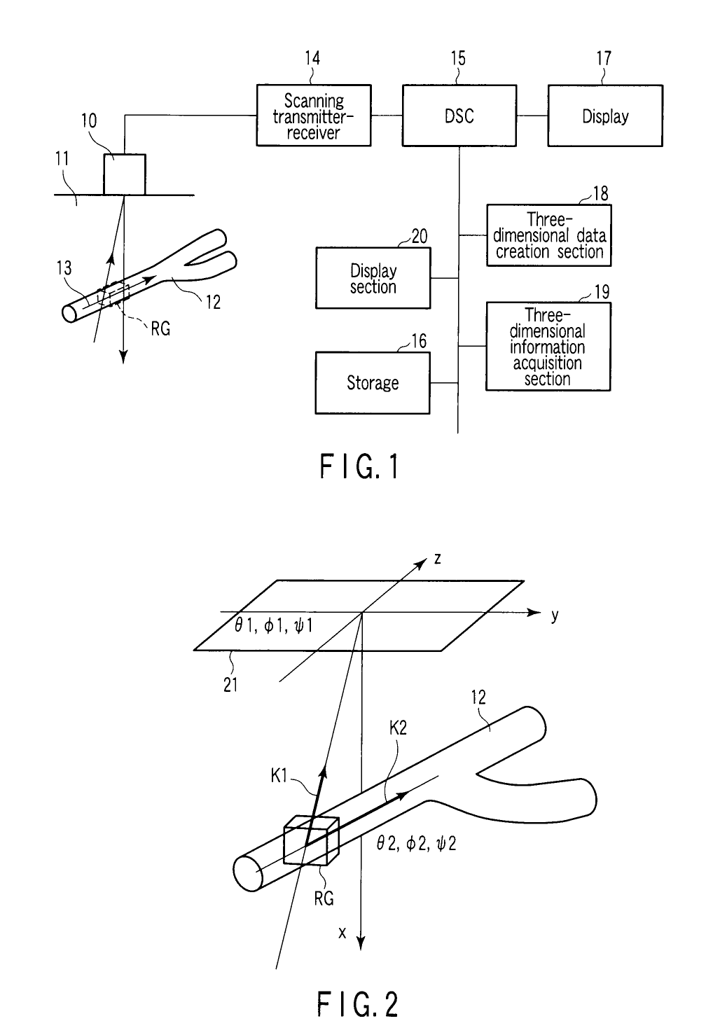

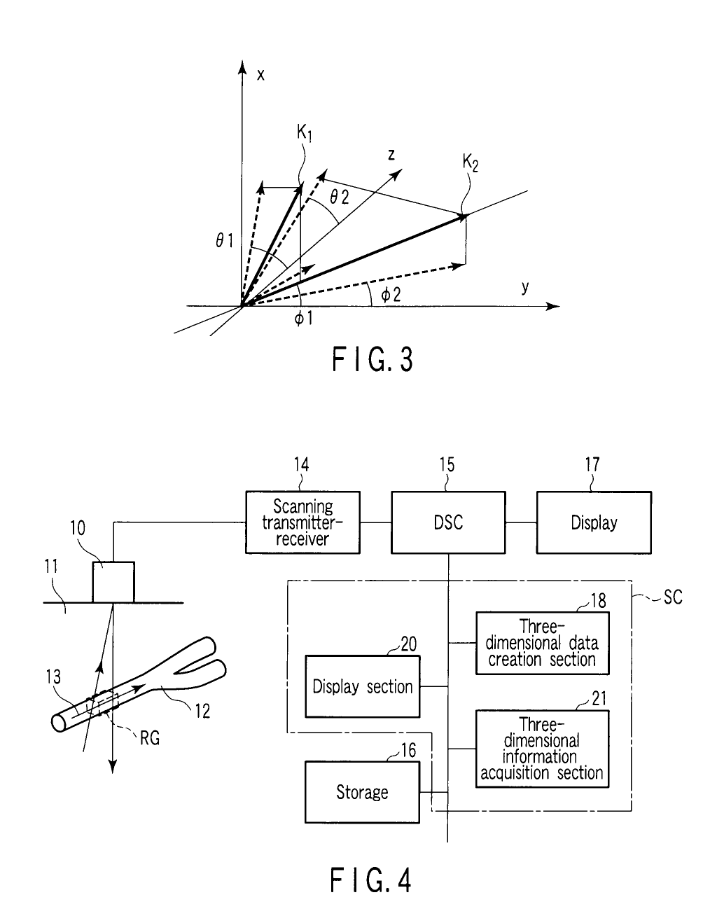

Namely, as shown in FIG. 2 and FIG. 3, the ultrasonic probe 10 has a probe surface 21 with ultrasonic oscillators arranged on a two-dimensional plane. The vector directions of the received beam K₁ from the range gate RG are assumed as θ₁, φ₁ and φ₁, respectively. The vector directions of the blood vessel in its traveling direction are assumed as θ₂, φ₂ and φ₂, respectively. The three-dimensional information acquisition section 19 acquires fluid vector data K₂ as three-dimensional fluid information in the range gate RG, based on a velocity of blood flow expressed by the vector of the received beam K₁, vector directions θ₁, φ₁ and φ₁ of the received beam K₁, vector directions θ₂, φ₂ and φ₂ of the blood vessel 13 in its traveling direction. The fluid vector data K₂ indicates a three-dimensional flow direction of the sample 13 such as a blood flow, and a flow rate of blood of the sample 13 such as a blood flow.

Next, an explanation will be given on the operation of the apparatus configured as explained above.

In the ultrasonic probe 10, the ultrasonic oscillators are electronically scanned by the scanning transmitter-receiver 14, the ultrasonic oscillators are sequentially driven, and a multiple ultrasonic beam is emitted. The multiple ultrasonic beam is transmitted to a range gate RG in the living body 11 such as a human body, for example. The ultrasonic probe 10 receives a wave reflected from an area including the range gate RG, and outputs a signal from each ultrasonic oscillator. The scanning transmitter-receiver 14 detects a Doppler signal from the output signal from each ultrasonic oscillator when receiving the wave reflected from the range gate RG.

The DSC 15 converts a Doppler signal output from the scanning transmitter-receiver 14 to a digital signal, and stores such signal in the storage 16 such as an image memory. The DSC 15 reads the digital Doppler signal stored in the storage 16 according to the scanning of the display 17. The DSC 15 converts the digital Doppler signal to an analog signal, and displays an ultrasonic image of the range gate RG in the living body 11 such as a human body, real time in the display 17. Namely, the three-dimensional data creation section 18 of the DSC 15 converts the Doppler signal output from the scanning transmitter-receiver 14 to a digital signal, and stores the digital Doppler signal for a preset scanning period it in the storage 16. As a result, the three-dimensional image data creation section 18 acquires a plurality of sectional image data (stack data). By reconfiguring the obtained sectional image data, the three-dimensional image data creation section 18 creates three-dimensional ultrasonic image data (volume data) of the range gate RG in the living body 11 such as a human body.

The three-dimensional information acquisition section 19 acquires three-dimensional fluid information including at least three-dimensional flow direction of the sample 13 included in the three-dimensional ultrasonic image data of a specific area, or the range gate RG in the living body 11 such as a human body, created by the three-dimensional data creation section 18.

Namely, as shown in FIG. 2 and FIG. 3, the three-dimensional information acquisition section 19 acquires fluid vector data K₂ indicating a three-dimensional flow direction and flow rate of the sample 13 such as a blood flow in the range gate RG as three-dimensional fluid information, based on a velocity of blood flow expressed by the vector of the received beam K₁, vector directions θ₁, φ₁ and φ₁ of the received beam K₁, and directions θ₂, φ₂ and φ₂ of the blood vessel 13 in its traveling direction.

As explained above, according to the first embodiment, the ultrasonic probe 10 emits a multiple ultrasonic beam to the range gate RG including the sample 13 such as blood flowing in the blood vessel 12 in the living body 11 such as a human body, receives waves reflected from parts arranged at very small intervals in the sample 13, and acquires the fluid vector data K₂ indicating a three-dimensional flow direction and flow rate of the sample 13 such as a blood flow in the range gate RG, based on the Doppler signal output from the ultrasonic probe 10. Therefore, the flow velocity and direction of a sample such as a blood flow can be exactly acquired.

Next, a second embodiment of the invention will be explained with reference to the accompanying drawings. The same parts as in FIG. 1 are given the same reference numbers, and detailed explanation on these same parts will be omitted.

FIG. 4 is a block diagram of an ultrasonic Doppler diagnostic apparatus. The ultrasonic probe 10 emits a multiple ultrasonic beam pulse consisting of a plurality of beams to a specific area (hereinafter called a range gate RG) including the sample 13 that is fluid such as blood flowing in the blood vessel 12 in the living body 11 such as a human body, and receives a wave reflected from a range gate RG. The ultrasonic probe 10 uses a scan beam so-called n*m. The values of n and m are 2 or more. Here, a scan beam of 2*2 (2 by 2) is used.

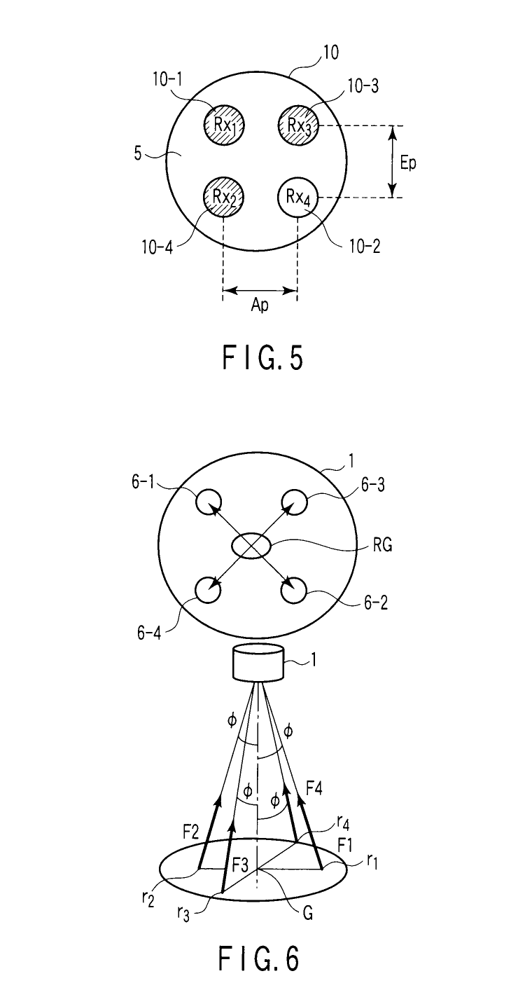

The ultrasonic probe 10 comprises ultrasonic oscillators (Tx and Rx) arranged on a two-dimensional plane. The ultrasonic probe 10 transmits a multiple ultrasonic beam, and receives waves reflected from the ultrasonic oscillators (Tx and Rx). FIG. 5 is a schematic diagram of a two-dimensional probe surface of the ultrasonic probe 10. The ultrasonic probe 10 can receive a wave reflected from the range gate RG by using four ultrasonic oscillators 10-1 to 10-4 (Rx 1 to Rx 4), for example, of the ultrasonic oscillators arranged on the two-dimensional plane. When the ultrasonic oscillators 10-1, 10-3 and 10-4 are used, an interval between the ultrasonic oscillators 10-1 and 10-4 is assumed as an elevation pitch Ep. An interval between the ultrasonic oscillators 10-1 and 10-3 is assumed as an azimuth pitch Ap.

The digital scan converter (hereinafter called a DSC) 15 converts a Doppler signal output from the scanning transmitter/receiver 14 to a digital signal, and reads the digital Doppler signal by a signal processor SC according to the scanning of the display 17. The DSC 15 converts the read digital Doppler signal to an analog signal, and displays an ultrasonic image of the range gate RG real time in the display 17. The range gate RG, as explained above, includes the sample 13 such as blood flowing in the blood vessel 12 in the living body 11 such as a human body. The digital Doppler signal is stored in the storage 16 such as an image memory.

The signal processor SC has functions as a three-dimensional image data creation section 18, a three-dimensional information acquisition section 21, and a display section 20.

The three-dimension information acquisition section 21 acquires three-dimensional fluid information including at least three-dimensional flow direction of the sample 13 included in the three-dimensional ultrasonic image data of a specific area, or the range gate RG in the living body 11 such as a human body, created by the three-dimensional data creation section 18. Namely, the three-dimensional information acquisition section 21 acquires a velocity (a velocity of blood flow), an azimuth angle, and an elevation angle of the sample 13 such as a blood flow as three-dimensional fluid information, based on fluid vector data indicating a three-dimensional flow direction and blood flow rate of the sample 13 such as a blood flow included in the three-dimensional ultrasonic image data.

Now, an explanation will be given on calculation of a norm of fluid vector data indicating the blood flow rate in the sample 13 such as a blood flow. An angle formed by a direction of ultrasonic beam and a flow direction of the sample 13 such as a blood flow (hereinafter called a blood flow direction) is called a Doppler angle. In measurement of a velocity of blood flow by the ultrasonic Doppler method, a Doppler deviation frequency to be detected is proportional to the product of a cosine of a velocity of blood flow and a Doppler angle, and depends on a Doppler angle. Obtaining an absolute value of a velocity of blood flow by measuring a Doppler angle is called a Doppler angle correction. The Doppler angle correction is used for calculation of a norm (a velocity of blood flow) of the fluid vector data indicating the blood flow rate in the sample 13 such as a blood flow. The Doppler angle correction will be explained.

As shown in FIG. 6, the angles formed by four directions in the elevation and azimuth, crossing the range gate RG including the sample 13 such as a blood flow, are the same φ. The range gate RG including the sample 13 such as a blood flow exists at the center of the four ultrasonic beams. Blood is assumed to be uniformly flowing in the range gate RG.

As the angle φ (hereinafter called the elevation angle) formed by the four directions in the elevation and azimuth is small, the distance from the center G to the reflection points r₁ to r₄ of the received beam F₁ to F₄ is assumed to be the same depending upon the swing angle when scanning an ultrasonic beam. The elevation angle φ is previously known.

Directions of received beams F₁ to F₄ are assumed to be faced in the same direction even at the center of the range gate RG. The received beams F₁ to F₄ are to be expressed by a vector.

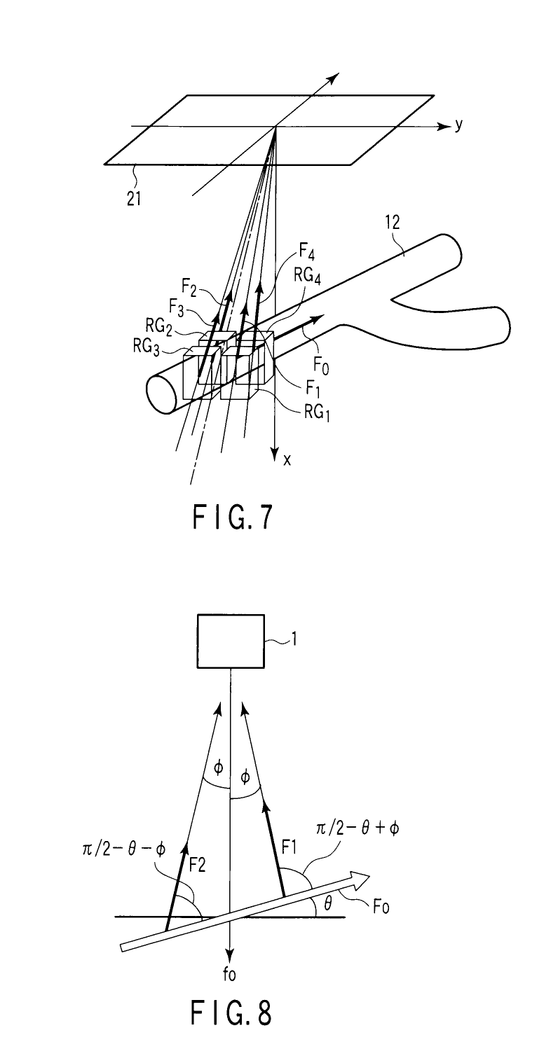

FIG. 7 shows the relationship between the received beam F₁ to F₄ and the range gate RG when a multiple ultrasonic beam is transmitted from the ultrasonic probe 10. The range gate RG comprises a plurality of part set at very small intervals on the blood vessel 12, for example four small range gates RG1 to RG4. Received beams F₁ to F₄ are waves reflected from small range gates RG1 to RG4, respectively.

First, an explanation will be given on a method of calculation on a two-dimensional cross section by referring to FIG. 8.

The received beams F₁ to F₄ are received by the ultrasonic oscillators 10-1 to 104, respectively at four locations in the ultrasonic probe 10.

The scanning transmitter-receiver 14 electronically scans the ultrasonic oscillators of the ultrasonic probe 10, and detects a Doppler signal from the output signal from the ultrasonic oscillators 10-1 to 104, respectively. The three-dimensional information acquisition section 21 performs the following calculation based on a Doppler signal received by the ultrasonic oscillators 10-1 to 104, respectively.

Scalar of the received beams F₁ to F₄ are assumed as f₁ to f₄, respectively, and a fluid vector indicating a blood flow rate in the sample 13 such as a blood flow, or an unknown blood flow vector, is assumed as F₀. f₀ indicates a velocity of blood flow that is the scalar of the blood flow vector F₀. An angle θ is assumed as an azimuth. Therefore,

f₁=f₀*sin(π/2−θ+φ)

f₂=f₀*sin(π/2−θ−φ)

Expressing by other equations,

f₁=f₀*cos(θ−φ)

f₂=f₀*cos(θ+φ)

The above equations are developed as follows.

f₁=f₀*(sin θ*cos φ−cos θ*sin φ)

f₂=f₀*(sin θ*cos φ+cos θ*sin φ)

However,

tan θ={(f₁+f₂)/(f₂−f₁)}*tan φ

The azimuth θ can be obtained by the following equation.

θ=tan⁻¹{(f₁+f₂)/(f₂−f₁)}*tan φ

The flow velocity f₀ of the sample 13 such as a blood flow after the angle correction can be obtained by the following equation.

$$f 0 = \frac{1}{2} * \sqrt{\frac{{( f 2 + f 1 )}^{2}}{{cos}^{2} φ} + \frac{{( f 2 - f 1 )}^{2}}{{sin}^{2} φ}}$$

Developing the above equation as a three-dimensional equation,

$$θ a = \frac{1}{2} \sqrt{\frac{{( f 2 + f 1 )}^{2}}{{cos}^{2} φ} + \frac{{( f 2 - f 1 )}^{2}}{{sin}^{2} φ}} θ a = {tan}^{- 1} ( \frac{f 1 + f 2}{f 2 - f 1} * tan φ )$$

$$fe = \frac{1}{2} \sqrt{\frac{{( f 4 + f 3 )}^{2}}{{cos}^{2} φ} + \frac{{( f 4 - f 3 )}^{2}}{{sin}^{2} φ}} θ e = {tan}^{- 1} ( \frac{f 4 + f 3}{f 4 - f 3} * tan φ )$$

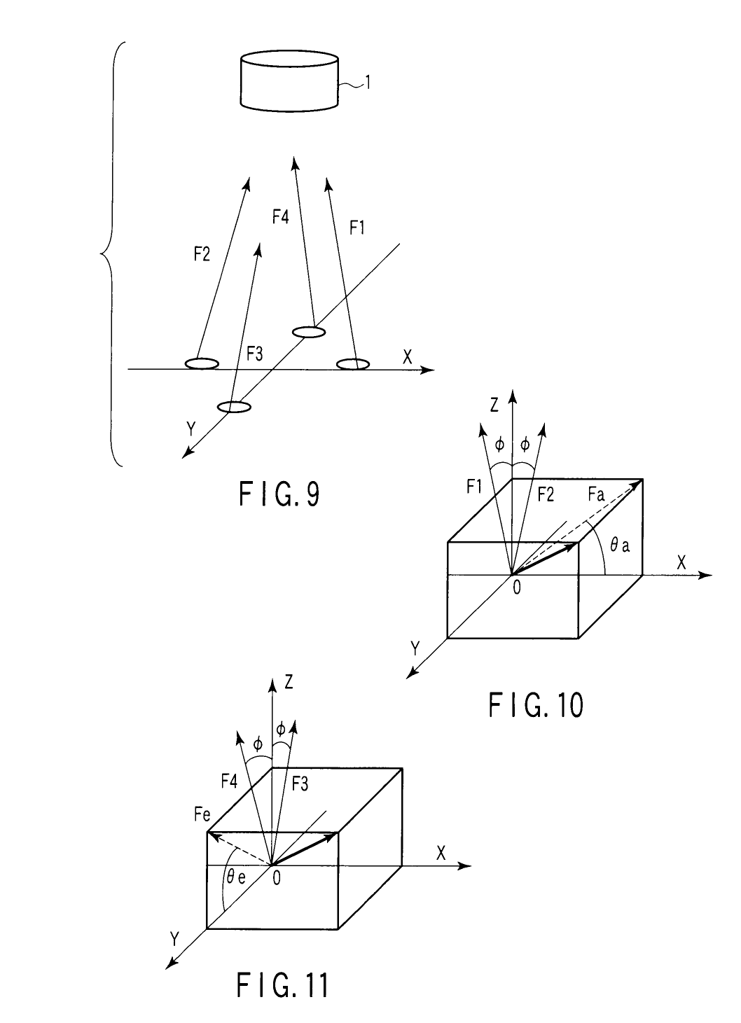

Namely, as shown in FIGS. 9 to 11, a cross section in the azimuth direction (X-Z plane) is calculated from the received beams F₁ and F₂, and a projection vector of a cross section in the elevation direction (Y-Z plane) is calculated from the received beams F₃ and F₄ by using a two-dimensional method.

As a result, the flow velocity fₒ of the three-dimensional blood flow vector F₀ can be obtained.

\vec{f0}=(fa*cos θa,fe*cos θe,fe*sin θe)

or (fa*cos θa,fe*cos θe,fa*sin θa)

|f0|=\sqrt{fe²+(fa*cos θa)²} or \sqrt{fa²+(fe*cos θe)²}

Therefore, the three-dimensional information acquisition section 21 acquires the velocity of blood flow fₒ, azimuth θ, and elevation angle φ of the sample 13 such as a blood flow as three-dimensional fluid information, which are expressed by the three-dimensional blood flow vector Fₒ started from the range gate RG.

The elevation angle φ is previously known. In this case, the three-dimensional information acquisition section 21 uses a multiple ultrasonic beam such as a 2 by 2 scan beam, and acquires the velocity of blood flow fₒ, azimuth θ, and elevation angle φ of the sample 13 such as two or more blood flows, by two or more beam.

The DSC 15 converts a Doppler signal output from the scanning transmitter-receiver 14 to a digital signal, and sends such signal to the signal processor. The ultrasonic probe 10 comprises ultrasonic oscillators arranged on a two-dimensional plane. As each ultrasonic oscillator sends a multiple ultrasonic beam, and receives a reflected wave, a reception delay adder circuit as described below is provided.

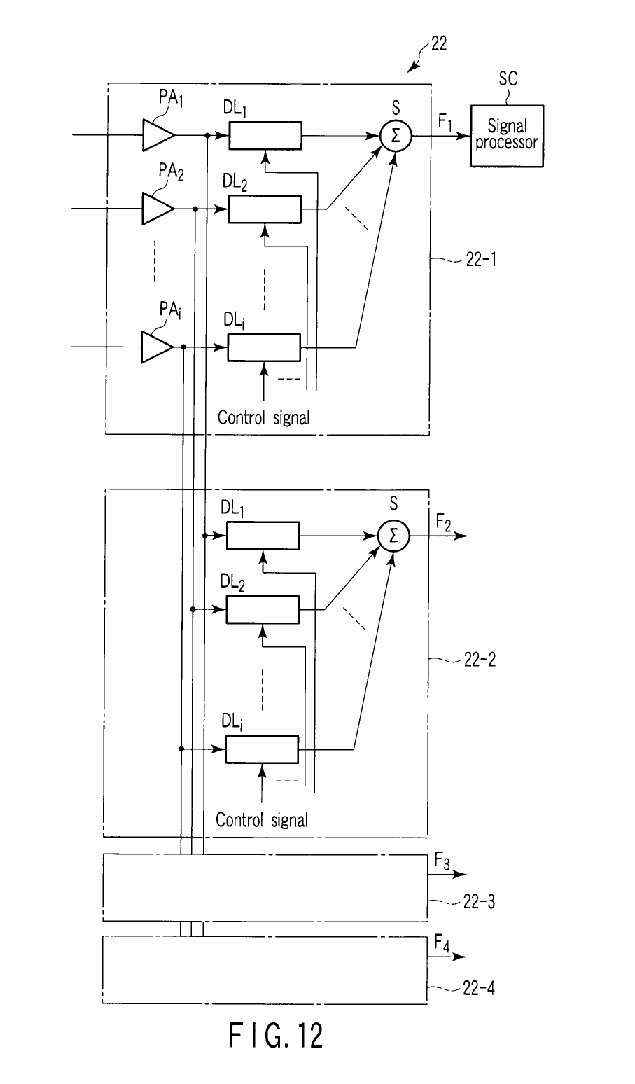

FIG. 12 is a block diagram of a reception delay adder circuit 22 using a delay line. The reception delay adder circuit 22 comprises four channels of reception delay adder circuit 22-1 to 22-4, for example, corresponding to the received beams F₁ to F₄ when a multiple ultrasonic beam is transmitted from the ultrasonic probe 10. The reception delay adder circuit 22 is provided with preamplifiers PA₁ to PAᵢ. The preamplifiers PA₁ to PAᵢ are provided corresponding to the ultrasonic oscillators of the ultrasonic probe 10, and used to amplify a Doppler signal output from each ultrasonic oscillator.

Namely, the preamplifiers PA₁ to PAᵢ correspond to the number i of the arranged ultrasonic oscillators. The output terminals of preamplifiers PA₁ to PAᵢ are connected to delay circuits DL₁ to DLᵢ, respectively. The delay circuits DL₁ to DLᵢ are controlled by a control signal received from a main controller in the DSC 15, for example. The delay circuits DL₁ to DLᵢ compensate for a delay between the beginning and end of one scan of a multiple ultrasonic beam in the ultrasonic probe 10, and creates one three-dimensional ultrasonic image data. The delay circuits DL₁ to DLᵢ are set to different delay time according to the scanning positions of a multiple ultrasonic beam. The output terminals of the delay circuit DL₁ to DLᵢ are connected to an adder S. The adder S adds the outputs of the delay circuits DL₁ to DLᵢ, and sends the added outputs to the signal processor SC.

The reception delay adder circuit 22-2 is provided with delay circuits DL₁ to DLᵢ. The delay circuits DL₁ to DLᵢ are controlled by a control signal received from a main controller in the DSC 15, for example. The delay circuits DL₁ to DLᵢ compensate for a delay between the beginning and end of one scan of a multiple ultrasonic beam in the ultrasonic probe 10, and creates one three-dimensional ultrasonic image data. The delay circuits DL₁ to DLᵢ are set to different delay time according to the scanning positions of a multiple ultrasonic beam. The output terminals of the delay circuit DL₁ to DLᵢ are connected to the adder S. The adder S adds the outputs of the delay circuits DL₁ to DLᵢ, and sends the added outputs to the signal processor SC. The signal processor SC may be provided exclusively for the reception delay adder circuit 22-2.

As in the reception delay adder circuit 22-2, the reception delay adder circuits 22-3 and 22-4 are provided with delay circuits DL₁ to DLᵢ. The configurations of the reception delay adder circuits 22-3 and 22-4 are the same as the reception delay adder circuit 22-2, and an illustration of a concrete configuration is omitted in FIG. 12. The delay circuits DL₁ to DLᵢ are controlled by a control signal received from a main controller in the DSC 15, for example. The delay circuits DL₁ to DLᵢ compensate for a delay between the beginning and end of one scan of a multiple ultrasonic beam in the ultrasonic probe 10, and creates one three-dimensional ultrasonic image data. The delay circuits DL₁ to DLᵢ are set to different delay time according to the scanning positions of a multiple ultrasonic beam. The output terminals of the delay circuit DL₁ to DLᵢ are connected to the adder S. The adder S adds the outputs of the delay circuits DL₁ to DLᵢ, and sends the added outputs to the signal processor SC. The signal processor SC may be provided exclusively for the reception delay adder circuits 22-2 and 22-4.

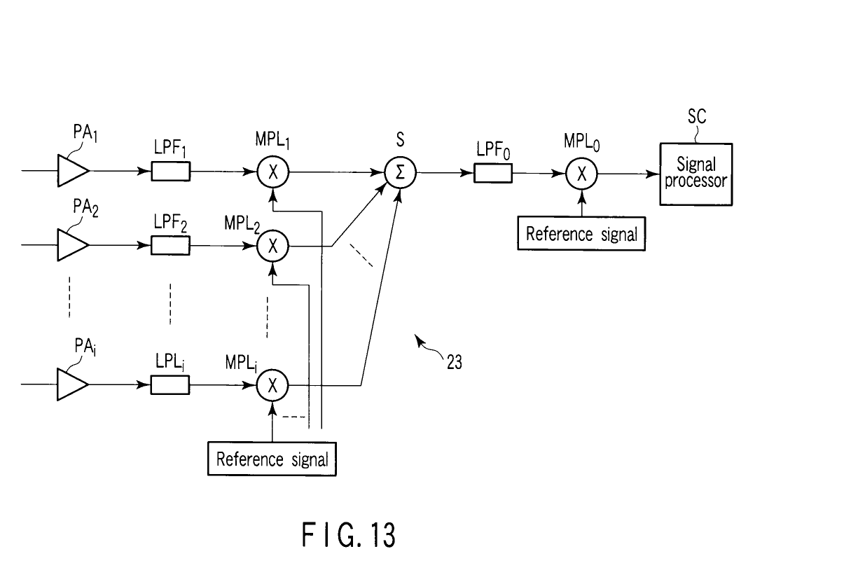

FIG. 13 is a block diagram of a reception delay adder circuit 23 using a multiplier. The output terminals of amplifiers PA₁ to PAᵢ are connected to multipliers MPL₁ to MPLᵢ through low-pass filters LPF₁ to LPFᵢ, respectively. The multipliers MPL₁ to MPLᵢ receive a reference signal from a main controller, and multiply a Doppler signal passing through each low-pass filter LPF₁ to LPFᵢ by a reference signal. The output terminals of the multipliers MPL₁ to MPLi are connected to the adder S. The adder S adds the outputs of the multipliers MPL₁ to MPLᵢ. The output terminal of the adder S is connected to a multiplier MPL₀ through a low-pass filter LPF₀. The multiplier MPL₀ multiplies the output signal of the low-pass filter LPF₀ by a reference signal, and sends the product to the signal processor SC.

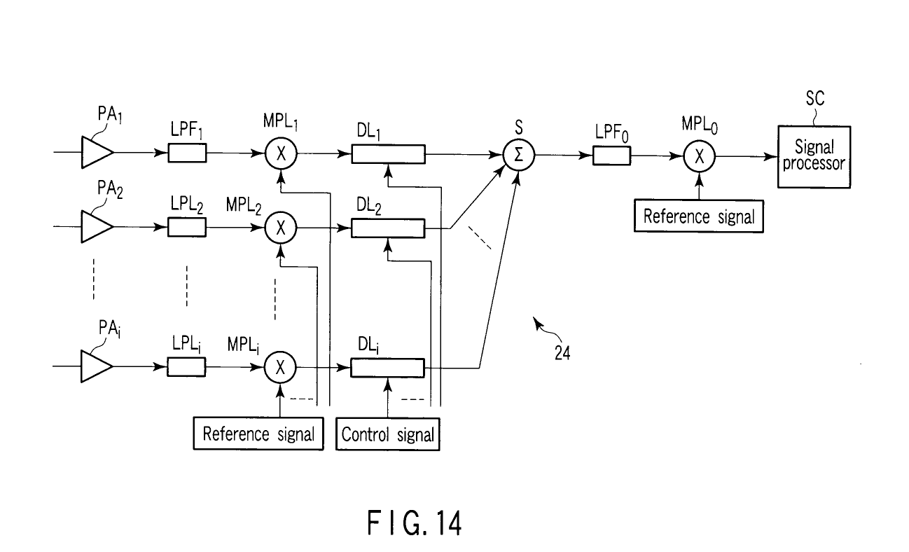

FIG. 14 is a block diagram of a reception delay adder circuit 24 combining a delay circuit and a multiplier. The same parts as in FIG. 12 and FIG. 13 are given the same reference numbers, and detailed explanation on these same parts will be omitted. The output terminals of amplifiers PA₁ to PAᵢ are connected to multipliers MPL₁ to MPLᵢ through low-pass filters LPF₁ to LPFᵢ, respectively. The output terminals of the multipliers MPL₁ to MPLi are connected to the adder S through delay circuits DL₁ to Dlᵢ. The output terminal of the adder S is connected to a multiplier MPL₀ through the low-pass filter LPF0. The multiplier MPL₀ multiplies the output signal of the low-pass filter LPF₀ by a reference signal, and sends the product to the signal processor SC.

A display section 20 displays a velocity of blood flow f₀, azimuth θ and elevation angle φ of the sample 13 such as two or more blood flows, which are acquired by the three-dimensional acquisition section 21 as two or more beam, in the display 10. The display section 20 displays the three-dimensional ultrasonic image data of the range gate RG including the sample 13 such as a flood flow flowing in the blood vessel 12, for example, which are created by the three-dimensional image data creation section 18.

The display section 20 displays a spectrum, which expresses a velocity component fₒ of a blood flow by assuming a blood flow going to the ultrasonic probe 10 as positive and a flood flow leaving from the ultrasonic probe 10 as negative, according to a Doppler deviation frequency based on a Doppler signal, in the display 17. In this case, as the three-dimensional information acquisition section 21 acquires the velocity of blood flow f₀ of the sample 13 such as a blood flow from two or more beams, IQ data in which these velocity of blood flow fₒ are added is used. The Doppler angle correction is performed by 1/cos θ/cos φ. When displaying the three-dimensional ultrasonic image data of the range gate RG including the sample 13 such as a blood flow in the display 10, the display section 20 displays an angle compensation mark in the display 10 on a volume and multi-plane corresponding to the position of the range gate RG on the screen of the display 10, according to the azimuth θ and elevation angle φ.

The display section 20 displays the sample 13 such as a blood flow in the display 10 in colors corresponding to the velocity of blood flow fₒ, azimuth θ and elevation angle φ acquired by the three-dimensional acquisition section 21. Namely, the display section 20 uses the Munsell color system. The Munsell color system consists of Munsell hue H, Munsell chroma S and Munsell value I. Munsell hue H and Munsell chroma S and Munsell value I form an HSI color space.

First, the display section 20 converts the velocity of blood flow fₒ, azimuth θ and elevation angle φ of the sample 13 such as a blood flow, to information about the HIS color space by using a previously set HIS conversion table. The HIS conversion table consists of the following equations, for example. In such equations, a1-a3, b1-b3, and c1-c3 are optional constants. The constants a1-a3, b1-b3, and c1-c3 can be varied according to the colors corresponding to the velocity of blood flow fₒ, azimuth θ and elevation angle φ of the sample 13 such as a blood flow, when displaying in the display 10.

$$[ \begin{matrix}H \\ S \\ I\end{matrix} ] [ \begin{matrix}{a}_{1} & {a}_{2} & {a}_{3} \\ {b}_{1} & {b}_{2} & {b}_{3} \\ {c}_{1} & {c}_{2} & {c}_{3}\end{matrix} ] [ \begin{matrix}θ \\ φ \\ f 0\end{matrix} ]$$

Next, the display section 20 converts the information about the HIS color space to RGB information by using a preset RGB conversion table. The RGB information consists of red (R), green (G) and blue (B).

Then, the display section 20 colors the three-dimensional ultrasonic image data of the range gate RG including the sample 13 such as a blood flow, according to the RGB information, and displays the colored three-dimensional ultrasonic image data in the display 10. As a result, the display 10 displays the color three-dimensional ultrasonic image data corresponding to the velocity of blood flow fₒ, azimuth θ and elevation angle φ of the sample 13 such as a blood flow.

Next, an explanation will be given on the operation of the apparatus configured as above described.

In the ultrasonic probe 10, the ultrasonic oscillators are electronically scanned by the scanning transmitter-receiver 14, the ultrasonic oscillators are sequentially driven, and a multiple ultrasonic beam is emitted. The multiple ultrasonic beam is transmitted to the range gate RG in the living body 2 such as a human body, for example. The ultrasonic probe 10 receives a wave reflected from an area including the range gate RG, and outputs a signal from each ultrasonic oscillator. The scanning transmitter-receiver 14 detects a Doppler signal from the output signal from each ultrasonic oscillator when receiving the wave reflected from the range gate RG.

Next, the DSC 8 converts a Doppler signal output from the scanning transmitter-receiver 14 to a digital signal, and stores the digital Doppler signal in the storage 9 such as an image memory. The DSC 8 reads the digital Doppler signal stored in the storage 9 according to the scanning of the display 10, converts the digital Doppler signal into an analog signal, and displays an ultrasonic image of the range gate RG in the living body 2 such as a human body, in real time on the display 10.

Namely, the three-dimensional data creation section 18 of the DSC 8 converts a Doppler signal output from the scanning transmitter-receiver 14 to a digital signal, and stores the digital Doppler signal for a preset scanning period in the storage 9, thereby obtaining two or more sectional image data (stack data). By reconfiguring the obtained sectional image data, the three-dimensional image data creation section 18 creates three-dimensional ultrasonic image data (volume data) of the range gate RG in the living body 2 such as a human body.

Next, the three-dimensional information acquisition section 21 acquires three-dimensional fluid information including at least three-dimensional flow direction of the sample 13 included in the three-dimensional ultrasonic image data of a specific area, or the range gate RG in the living body 11 such as a human body, created by the three-dimensional data creation section 18. Namely, the three-dimensional information acquisition section 21 acquires a velocity (a velocity of blood flow), azimuth and elevation angle of the sample 13 such as a blood flow as three-dimensional fluid information, based on fluid vector data indicating a three-dimensional flow direction and flow rate of the sample 13 such as a blood flow included in the three-dimensional ultrasonic image data. The three-dimensional information acquisition section 21 uses Doppler angle correction for calculation of a norm (a flood flow velocity) of the fluid vector data indicating the blood flow rate in the sample 13 such as a blood flow as above explained.

By using such Doppler angle correction, the three-dimensional information acquisition section 21 acquires the velocity of blood flow fₒ, azimuth θ, and elevation angle φ of the sample 13 such as a blood flow as three-dimensional fluid information, which are expressed by the three-dimensional blood flow vector Fₒ started from the range gate RG. In this case, the three-dimensional information acquisition section 21 uses a multiple ultrasonic beam such as a 2 by 2 scan beam, and acquires the velocity of blood flow fₒ, azimuth θ, and elevation angle φ of the sample 13 such as a blood flow of a plurality of blood flow, by two more beams.

A display section 20 displays the velocity of blood flow f₀, azimuth θ and elevation angle φ of the sample 13 such as blood flows, which are acquired by the three-dimensional acquisition section 21 as two or more beam, in the display 10. The display section 20 displays the three-dimensional ultrasonic image data of the range gate RG including the sample 13 such as a flood flow flowing in the blood vessel 12, for example, which are created by the three-dimensional image data creation section 18, in the display 10. The display section 20 displays a spectrum, which expresses a velocity component fₒ of blood flow by assuming a blood flow going to the ultrasonic probe 10 as positive and a flood flow leaving from the ultrasonic probe 10 as negative, according to a Doppler deviation frequency based on a Doppler signal, in the display 17.

The display section 20 displays the sample 13 such as a blood flow in the display 10 in colors corresponding to the velocity of blood flow fₒ, azimuth θ and elevation angle φ acquired by the three-dimensional acquisition section 21. Namely, the display section 20 converts the velocity of blood flow fₒ, azimuth θ and elevation angle φ of the sample 13 such as a blood flow, to information about the HIS color space by using a previously set HIS conversion table.

Next, the display section 20 converts the information about the HIS color space to RGB information by using a preset RGB conversion table.

Then, the display section 20 colors the three-dimensional ultrasonic image data of the range gate RG including the sample 13 such as a blood flow, according to the RGB information, and displays the colored three-dimensional ultrasonic image data in the display 10. As a result, the display 10 displays the color three-dimensional ultrasonic image data corresponding to the velocity of blood flow fₒ, azimuth θ and elevation angle φ of the sample 13 such as a blood flow.

As above described, according to the second embodiment, the ultrasonic probe 10 transmits a multiple ultrasonic beam, and the three-dimensional information acquisition section 21 acquires the velocity of blood flow f₀, azimuth θ and elevation angle φ of the sample 13 such as a blood flow as three-dimensional fluid information in the range gate RG. Therefore, the velocity of blood flow f₀, azimuth θ and elevation angle φ of the sample 13 such as a blood flow can be exactly acquired. As the elevation angle φ of each received beam F1 to F₄ is constant, even if the distance from the ultrasonic probe 10 to the sample 13 becomes long, the velocity of blood flow f₀, azimuth θ and elevation angle φ of the sample 13 such as a blood flow can be exactly acquired without decreasing the accuracy of Doppler angle correction.

The sample 13 such as a blood flow is displayed in the display 10 in colors corresponding to the velocity of blood flow fₒ, azimuth θ and elevation angle φ acquired by the three-dimensional acquisition section 21. Therefore, the velocity of blood flow fₒ, azimuth θ and elevation angle φ of the sample 13 such as a blood flow can be confirmed from the color of the three-dimensional ultrasonic image displayed in the display 10.

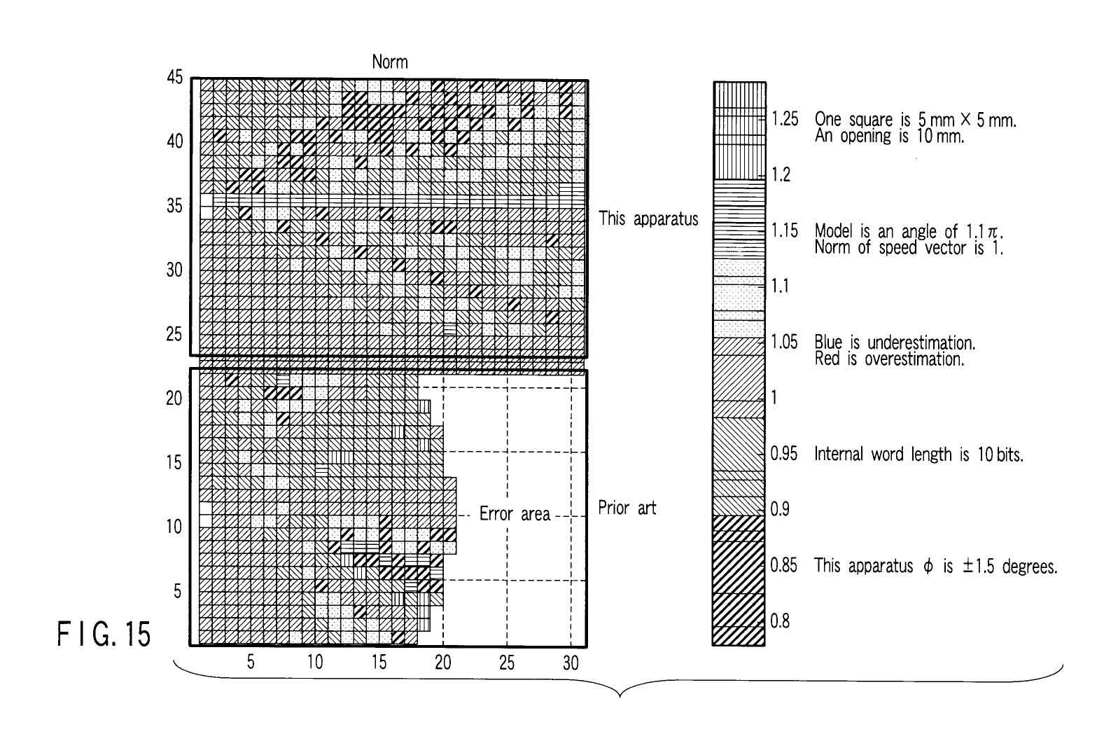

FIG. 15 shows a result of error evaluation simulation for estimation of the velocity fₒ of the example 13 such as a blood flow. The drawing shows the results of the error evaluation simulation by the first technology (the literature by Jorgen Arendet Jensen) and the error evaluation simulation by Dr. Jensen shown in FIG. 21. As the elevation angle φ becomes larger when a 2 by 2 scan beam is used, the accuracy of Doppler angle correction is increased. In the prior art, when the distance from the ultrasonic probe 10 to the sample 13 becomes long, the accuracy of Doppler angle correction is decreased, and an error may occur when a critical point is exceeded. Therefore, in the prior art, it is impossible to acquire exact velocity of blood flow fₒ, azimuth θ and elevation angle φ of the sample 13 such as a blood flow.

Further, as a multiple ultrasonic beam such as a 2 by 2 scan beam is used, the velocity of blood flow fₒ, azimuth θ and elevation angle φ of the sample 13 such as a blood flow can be acquired by two or more beams. For example, the average (f₀₁+f₀₂+ . . . +f₀ⱼ/j) of two or more blood flow velocities f₀₁, f₀₂, . . . , f₀ⱼ is displayed, Doppler angle correction is manually performed, and the displays of the velocity of blood flow fₒ, azimuth θ and elevation angle φ of the velocity compensated three-dimensional blood flow vector F₀ may be switched.

When the azimuth θ formed by a multiple ultrasonic beam sent from the ultrasonic probe 10 and a received beam becomes small, blood information necessary for color mapping in the range gate RG, for example, is more exactly reflected. In contrast, when the azimuth θ formed by a multiple ultrasonic beam sent from the ultrasonic probe 10 and a received beam becomes large, the measurement accuracy of the velocity of blood flow fₒ is increased.

Therefore, first of all, the ultrasonic probe 10 sets the distance to the sample 13 such as a blood flow short. The distance from the ultrasonic probe 10 to the sample 13 may be previously set, or optionally set. In this state, the ultrasonic probe 10 increases the azimuth θ formed by a multiple ultrasonic beam and a received beam, and acquires the velocity of blood flow f₀.

Then, the ultrasonic probe 10 sets the distance to the sample 13 long. The distance from the ultrasonic probe 10 to the sample 13 at this time may be previously set, or optionally set. In this state, the ultrasonic probe 10 increases the azimuth θ formed by a multiple ultrasonic beam and a received beam, and acquires the velocity of blood flow f₀.

Therefore, blood flow information necessary for color mapping in the range gate RG, for example, can be exactly measured, and the measurement accuracy of the velocity of blood flow fₒ is increased.

The present invention is not to be limited to the embodiment described herein. The invention may be modified by modifying the components in a practical stage without departing from its spirit and essential characteristics. The invention may be embodied in other forms by appropriately combining the components disclosed in the embodiment described herein. For example, some components may be deleted from the total components shown in the embodiment. Further, the components of different embodiment may be combined.

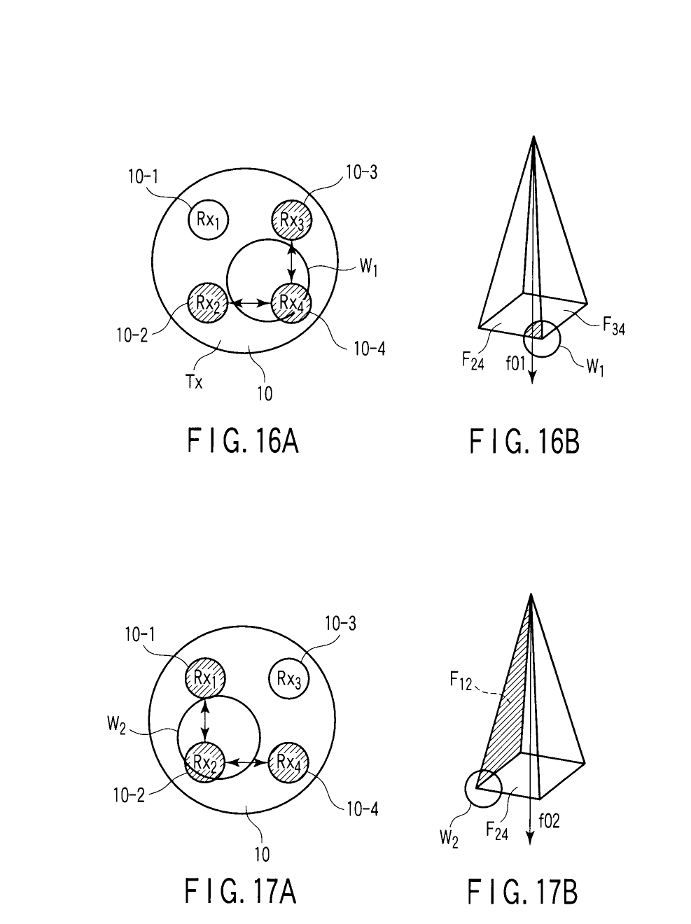

The ultrasonic oscillators (Rx 1 to Rx 4) 10-1 to 10-4 in the ultrasonic probe 10 may be used as shown in FIGS. 16A and 16B to FIGS. 19A and 19B, for example. FIG. 16A shows a measurement area W₁ of a flow velocity k in a blood flow direction, for example, as the sample 13 when the ultrasonic oscillators (Rx 2 to RX 4) 10-2 to 10-4 are used. FIG. 16B shows a transmitted multiple ultrasonic beam f₀₁ and received beams F₂₄ and F₃₄.

FIG. 17A shows a measurement area W₂ of a flow velocity in a blood flow direction, for example, as the sample 13 when the ultrasonic oscillators (Rx 1, Rx 2 and RX 4) 10-1, 10-2 and 10-4 are used. FIG. 17B shows a transmitted multiple ultrasonic beam f₀₂, and received beams F₂₄ and F₁₂.

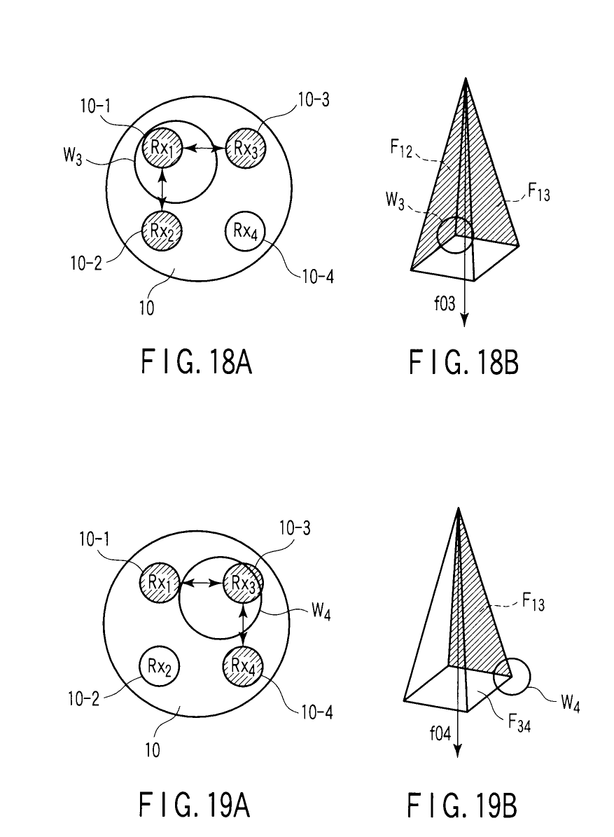

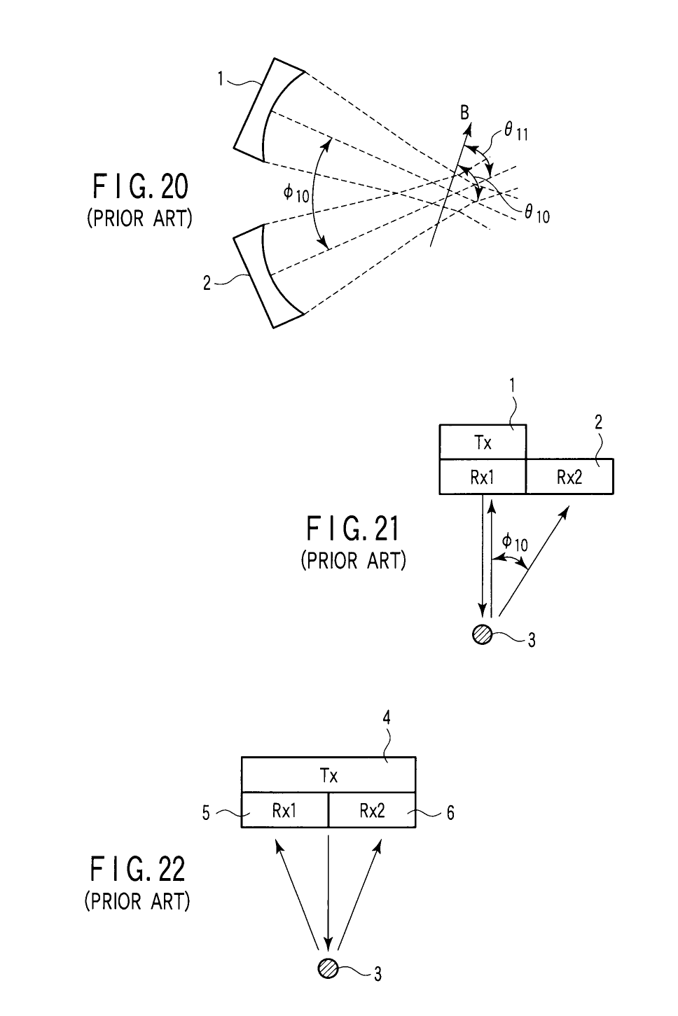

FIG. 18A shows a measurement area W₃ of a flow velocity in a blood flow direction, for example, as the sample 13 when the ultrasonic oscillators (Rx 1 to Rx 3) 10-1 to 10-3 are used. FIG. 18B shows a transmitted multiple ultrasonic beam f₀₃, and received beams F₁₂ and F₁₃.

FIG. 19A shows a measurement area W₄ of a flow velocity in a blood flow direction, for example, as the sample 13 when the ultrasonic oscillators (Rx 1, Rx 3 and Rx 4) 10-1, 10-3 and 10-4 are used. FIG. 19B shows a transmitted multiple ultrasonic beam f₀₄, and received beams F₁₃ and F₃₄.

When a velocity of blood flow in a range gate RG that is a part to measure a blood flow, for example by emitting an ultrasonic beam by using a pulse Doppler method (PWD), the shape of the range gate RG can be changed. In biplane color scanning in an ultrasonic diagnostic apparatus, or in a scanning method for acquiring two intersecting sectional images substantially real time, the thickness azimuth can be changed in the biplane color scanning.

The ultrasonic oscillators (Rx 1 to Rx 4) 10-1 to 10-4 in the ultrasonic probe 10 may be changed in the elevation angle φ to receive a wave reflected from a range gate RG. For example, when a 2 by 2 scan beam is used, when the elevation angle φ is increased, the accuracy in acquiring the velocity of blood flow fₒ, azimuth θ and elevation angle φ of the sample 13 such as a blood flow is increased.

The three-dimensional information acquisition section 18 may be configured to acquire fluid vector data indicating a three-dimensional flow direction and flow rate of the sample 13 for two or more parts based on a Doppler signal output from the ultrasonic probe 10, and to acquire the velocity of at least the sample 13 by adding and averaging these fluid vector data. For example, the three-dimensional information acquisition section 18 is configured to acquire the velocity of the sample 13 such as a blood flow, by adding and averaging the fluid vectors indicting the received beams F₁ to F₄ from four small range gates RG₁ to R₄ shown in FIG. 7. In color Doppler tomography, a blood velocity is indicated by color. For example, blood flow information is overlapped in colors on a monochrome B-mode image. Therefore, color blood information may be acquired from the velocity of the sample 13 such as a blood flow acquired by the three-dimensional acquisition section 18.

The three-dimensional information acquisition section 18 may acquire fluid vector data indicating a three-dimensional flow direction and blood flow rate of the sample 13 such as a blood flow in two or more parts, based on a Doppler signal output from the ultrasonic probe 10. For example, the acquisition section 18 acquires fluid vector indicating the received beams F₁ to F₄ from four small range gates RG₁ to RG₄ shown in FIG. 7. A velocity change is obtained by normalizing acceleration between these fluid vectors, or between the fluid vectors at one or both of elevation pitch and azimuth pitch. A parameter to evaluate a degree of dispersion may be obtained based on the velocity change, or color mapping of a speed distribution of the sample 13 such as a blood flow may be obtained from the evaluation parameter.

As a blood flow vector F₀ exists on the three-dimensional coordinates of a vector or three-dimensional ultrasonic image data, the vector F₀ can be displayed by a normal fluid postprocessor. For example, such data can be displayed as a contour plot, a vector plot in a flow line display, a modification diagram, a graph, or a particle trace in a flow track trace.

As elevation information is included, in addition to azimuth information, in the sectional image data obtained from three-dimensional ultrasonic image data acquired by an ultrasonic diagnostic apparatus including biplane scanning, an accurate norm (a velocity of blood flow) of fluid vector data indicating a blood flow rate of the sample 13 such as a blood flow can be obtained with compensation made for the thickness direction of the sectional image data.

Further, whether a blood flow is going to or leaving from the thickness direction can be displayed by color mapping in the sectional image data. For example, a blood flow leaving from or going to the ultrasonic probe 10 can be set to red or blue, and a blood flow leaving from or going to the thickness direction of sectional image data can be displayed in red or blue.

By using the velocity of blood flow fₒ, azimuth θ and elevation angle φ of the sample 13, a blood flow on intersecting planes can be displayed by a vector quantity, an arrow and a flow line, for example, according to each plane, upon display of biplane blue eyes.

What is claimed is:

1. An ultrasonic diagnostic apparatus comprising:
an ultrasonic probe which transmits a multiple ultrasonic beam to a sample flowing in a specific area, and receives a wave reflected from each part set at very small intervals in the sample; and
a three-dimensional information acquisition section which acquires three dimensional fluid information including at least a three-dimensional flow direction of the sample in the specific area based on a Doppler signal output from the ultrasonic probe.

2. The ultrasonic diagnostic apparatus according to claim 1, wherein the three-dimensional information acquisition section acquires the three dimensional fluid information based on at least the magnitude and direction of the reflected wave received by the ultrasonic probe based on the Doppler signal output from the ultrasonic probe.

3. An ultrasonic diagnostic apparatus comprising:
an ultrasonic probe which transmits a multiple ultrasonic beam to a sample flowing in a specific area, and receives waves reflected from parts adjacent to each other in the sample in the specific area; and
a three-dimensional information acquisition section which acquires three dimensional fluid information including at least a three-dimensional flow direction of the sample in the specific area based on a Doppler signal output from the ultrasonic probe.

4. The ultrasonic diagnostic apparatus according to claim 3, wherein the ultrasonic probe has ultrasonic oscillators arranged at equal pitches in the vertical and horizontal directions to receive the waves reflected from the parts, and
the ultrasonic oscillators receive the waves reflected from the parts at the same elevation angle.

5. The ultrasonic diagnostic apparatus according to claim 4, wherein the ultrasonic oscillators receive the wave reflected from the parts adjacent to each other at four locations at the same elevation angle.

6. The ultrasonic diagnostic apparatus according to claim 4, wherein the ultrasonic oscillators have a variable elevation angle to receive the reflected wave.

7. The ultrasonic diagnostic apparatus according to claim 3, wherein the three-dimensional information acquisition section acquires fluid vector data indicating a three-dimensional flow direction and flow rate of the sample based on the Doppler signal output from the ultrasonic probe, and acquires a flow velocity, azimuth and elevation angle of the sample as the three-dimensional fluid information based on the fluid vector data.

8. The ultrasonic diagnostic apparatus according to claim 3, wherein the three-dimensional information acquisition section acquires the three-dimensional fluid information based on at least the magnitude and direction of each wave reflected from the parts received by the ultrasonic probe based on the Doppler signal outputs corresponding to the parts output from the ultrasonic probe.

9. The ultrasonic diagnostic apparatus according to claim 3, wherein the three-dimensional information acquisition section acquires fluid vector data indicating a three-dimensional flow direction and flow rate of the sample for each of the parts based on the Doppler signal output from the ultrasonic probe, and acquires at least a velocity of the sample by adding and averaging the fluid vector data.

10. The ultrasonic diagnostic apparatus according to claim 3, wherein the three-dimensional information acquisition section acquires fluid vector data indicating a three-dimensional flow direction and flow rate of the sample for each of the parts based on the Doppler signal output from the ultrasonic probe, acquires a velocity change by normalizing the fluid vector data at each pitch in the vertical and horizontal directions in which the ultrasonic oscillators are arranged, and makes color mapping of a velocity distribution of the sample based on the velocity change.

11. The ultrasonic diagnostic apparatus according to claim 3, wherein a three-dimensional ultrasonic image of an area including the sample is obtained by one of a pulse Doppler method and a color Doppler tomography.

12. The ultrasonic diagnostic apparatus according to claim 7, further comprising a display section which displays the sample in colors corresponding to the velocity, azimuth and elevation angle of the sample acquired by the three-dimensional acquisition section.

13. The ultrasonic diagnostic apparatus according to claim 1 or 3, wherein the ultrasonic probe increases an azimuth of the multiple ultrasonic beam as the distance to the sample is decreased, and decreases the azimuth of the multiple ultrasonic beam as the distance to the sample is increased.

14. A method of measuring a velocity with ultrasonic waves comprising:
transmitting a multiple ultrasonic beam from an ultrasonic probe to a sample flowing in a specific area, and receiving a wave reflected from each part set at very small intervals in the sample; and
acquiring three-dimensional fluid information including at least a three-dimensional flow direction of the sample in the specific area based on a Doppler signal output from the ultrasonic probe.

15. The method of measuring a velocity with ultrasonic waves according to claim 14, wherein each wave reflected from each part adjacent to each other in the sample in the specific area is received by the ultrasonic probe.
